(19) United States
(12) Patent Application Publication
(10) Pub. No.: US 2026/0046959 A1
(43) Pub. Date: Feb. 12, 2026

(54) MEASUREMENT DEVICE, INFORMATION TERMINAL, CONTROL METHOD, AND CONTROL RECORDING MEDIUM

(71) Applicant: OMRON HEALTHCARE Co., Ltd., Kyoto (JP)

(72) Inventors: Yasunori FUKUDA, Kyoto (JP); Tetsuzo OKAZAKI, Kyoto (JP)

(21) Appl. No.: 19/363,177

(22) Filed: Oct. 20, 2025

Related U.S. Application Data
(63) Continuation of application No. PCT/JP2024/001172, filed on Jan. 17, 2024.

(30) Foreign Application Priority Data
Jun. 7, 2023 (JP) 2023-093847

(51) Int. Cl. H04W 76/14; G16H 40/63; H04W 76/30

(52) U.S. Cl. CPC H04W 76/14; G16H 40/63; H04W 76/30

(57) ABSTRACT

A measurement device, an information terminal, a control method and a control program that can reduce power consumption. A measurement device that can measure biological information. The measurement device includes a wireless communication unit and a controller. The wireless communication unit is configured to transmit the measurement data of the biological information to an information terminal. Before an instruction to measure the biological information, the controller performs control to wirelessly transmit an advertise signal and wirelessly connect to the information terminal that made a connection request in response to the advertise signal, and at a time of measuring the biological information, in a case where a subject is different from a user a of the information terminal, the controller controls the wireless communication unit to disconnect from the information terminal and wirelessly connect to the information terminal of the subject.

### CROSS-REFERENCE TO RELATED APPLICATIONS

This application is the U.S. national stage application filed pursuant to 35 U.S.C. 365(c) and 120 as a continuation of International Patent Application No. PCT/JP2024/001172, filed Jan. 17, 2024, which application claims priority to, Japanese Patent Application No. 2023-093847, filed Jun. 7, 2023, which applications are incorporated herein by reference in their entireties.

### TECHNICAL FIELD

The present invention relates to a measurement device, an information terminal, a control method, and a control program (i.e., a control recording medium).

### BACKGROUND ART

A known system includes a smartphone and an electronic terminal that are communicably connected via Bluetooth Low Energy (BLE) (registered trademark) (Patent Document 1). Another known information processing system includes an information processing device including a smartphone or the like and a health device including a pedometer, an activity meter, a heart rate monitor, or the like and establishes a communication connection by exchanging an address, an authentication key, an encryption key, or the like and acquires measurement data via Bluetooth (registered trademark) or the like (Patent Document 2).

### CITATION LIST

### Patent Literature

Patent Document 1: JP 2019-136136 A
Patent Document 2: JP 2015-159979 A

### SUMMARY OF INVENTION

### Technical Problem

According to the system of Patent Document 1, an electronic terminal on the advertisement transmission side and a smartphone on the advertisement reception side establish a communication connection four times a day at predetermined times, transmit information measured by the electronic terminal to the smartphone, and thereafter disconnect the communication. The number of times of synchronization of the two is not limited to four times a day, and the trigger for synchronization is not limited to time.

According to the information processing system of Patent Document 2, with a normal operation mode and a power saving mode being set, so that the power consumed for advertising in the power saving mode is less than the power consumed in the normal operation mode, as timing control information, the cycle and time for advertising and the cycle and time for measuring time can be set for each mode.

Although it is possible to reduce power consumption by limiting the transmission timing for advertising, the opportunity for connection is likewise restricted by limiting the timing. However, Patent Document 1 and Patent Document 2 do not describe processing for restricting opportunity for connection. Thus, there is room for further improvement in the transmission of advertising and the reduction of power consumption.

The present invention has been made in view of such circumstances in one aspect, and an object thereof is to provide a measurement device, an information terminal, a control method, and a control program that can reduce power consumption.

### Solution to Problem

The present invention employs the following configurations to solve the above-described problems.

(1) A measurement device that can measure biological information including: a wireless communication unit configured to transmit measurement data of the biological information to an information terminal; and a control unit configured to control the wireless communication unit, wherein before an instruction to measure the biological information, the control unit performs control to wirelessly transmit an advertise signal and wirelessly connect to a first information terminal that made a connection request in response to the advertise signal, and at a time of measuring the biological information, in a case where a subject is different from a user of the first information terminal, the control unit performs control to disconnect from the first information terminal and wirelessly connect to a second information terminal of the subject.

According to (1), by transmitting the advertise signal and wirelessly connecting to the information terminal that made the connection request before receiving the instruction to measure the biological information, it is possible to reduce power consumption by stopping the transmission of an advertise signal with a transmission interval that cannot be made too long due to the specifications. Here, although it is necessary to transmit and receive packets at intervals of the Connection Interval in order to maintain the wireless connection, the Connection Interval can be made longer than the advertise signal, and thus the total power consumption can be reduced. In addition, in a case where the user of the information terminal, which was wirelessly connected before the instruction to measure the biological information is received, starts measuring, the measurement data can be transferred through the wireless connection, and thus, the convenience can be improved. In a case where the user, who is different from the user of the information terminal which was wirelessly connected before the instruction to measure the biological information is received, starts measuring, the wireless connection is established again with the information terminal of the user who started measuring and the measurement data can be transferred. Thus, it is possible to suppress a decrease in convenience caused by connecting wirelessly in advance.

(2) The measurement device according to (1), wherein at a time of wirelessly connecting to the second information terminal, the control unit wirelessly transmits an advertise signal including information indicating the subject.

According to (2), the information terminal of the user, who is different from the subject, can recognize from the advertise signal that the user different from the user of the information terminal is being measured, and thus can perform control so as not to make a connection request even if an advertise signal is detected. Thus, it is possible to suppress competition between the information terminal of the subject and the information terminal of the user different from the subject.

(3) The measurement device according to (1) or (2), wherein when a wireless connection with the first information terminal is established before measurement of the biological information, the control unit sets a signal transmission interval with the first information terminal to a first interval, and
at a time of wirelessly transmitting measurement data of the biological information to an information terminal of the subject, the control unit sets a signal transmission interval with an information terminal of the subject to a second interval shorter than the first interval.

According to (3), the first interval for transmitting the signal to the wirelessly connected first information terminal before measuring the biological information is set to be longer than the second interval for transmitting the signal when wirelessly transmitting the measurement data of the biological information to the information terminal of the subject, so that the number of times of signal transmission can be reduced, and power consumption can be reduced.

(4) An information terminal that can wirelessly communicate with a measurement device that can measure biological information, including:
a wireless communication unit configured to receive measurement data of the biological information from the measurement device; and a control unit configured to control the wireless communication unit, wherein in a case where an advertise signal wirelessly transmitted from the measurement device has been received, the control unit makes a connection request to the measurement device depending on whether or not information indicating a user different from a user of the information terminal is included in the advertise signal.

According to (4), since it is possible to recognize that the user different from the user of the information terminal is currently being measured on the basis of the information indicating the user included in the received advertise signal, it is possible to perform control so as not to perform a connection request by detecting the advertise signal. This can reduce unnecessary processing and reduce power consumption.

(5) A control method for a measurement device that can measure biological information and includes a wireless communication unit configured to transmit measurement data of the biological information to an information terminal and a processor configured to control the wireless communication unit, wherein before an instruction to measure the biological information, the processor performs control to wirelessly transmit an advertise signal and wirelessly connect to a first information terminal that made a connection request in response to the advertise signal; and at a time of measuring the biological information, in a case where a subject is different from a user of the first information terminal, the processor performs control to disconnect from the first information terminal and wirelessly connect to a second information terminal of the subject.

According to (5), by transmitting the advertise signal and wirelessly connecting to the information terminal that made the connection request before receiving the instruction to measure the biological information, it is possible to reduce power consumption by stopping the transmission of an advertise signal with a transmission interval that cannot be made too long due to the specifications. Here, although it is necessary to transmit and receive packets at intervals of the Connection Interval in order to maintain the wireless connection, the Connection Interval can be made longer than the advertise signal, and thus the total power consumption can be reduced. In addition, in a case where the user of the information terminal, which was wirelessly connected before the instruction to measure the biological information is received, starts measuring, the measurement data can be transferred through the wireless connection, and thus, the convenience can be improved. In a case where the user, who is different from the user of the information terminal which was wirelessly connected before the instruction to measure the biological information is received, starts measuring, the wireless connection is established again with the information terminal of the user who started measuring and the measurement data can be transferred. Thus, it is possible to suppress a decrease in convenience caused by connecting wirelessly in advance.

(6) A control program for a measurement device that can measure biological information and includes a wireless communication unit configured to transmit measurement data of the biological information to an information terminal and a processor configured to control the wireless communication unit, the control program causing the processor to execute processing wherein before an instruction to measure the biological information, control is performed to wirelessly transmit an advertise signal and wirelessly connect to a first information terminal that made a connection request in response to the advertise signal; and at a time of measuring the biological information, in a case where a subject is different from a user of the first information terminal, control is performed to disconnect from the first information terminal and wirelessly connect to a second information terminal of the subject.

According to (6), by transmitting the advertise signal and wirelessly connecting to the information terminal that made the connection request before receiving the instruction to measure the biological information, it is possible to reduce power consumption by stopping the transmission of an advertise signal with a transmission interval that cannot be made too long due to the specifications. Here, although it is necessary to transmit and receive packets at intervals of the Connection Interval in order to maintain the wireless connection, the Connection Interval can be made longer than the advertise signal, and thus the total power consumption can be reduced. In addition, in a case where the user of the information terminal, which was wirelessly connected before the instruction to measure the biological information is received, starts measuring, the measurement data can be transferred through the wireless connection, and thus, the convenience can be improved. In a case where the user, who is different from the user of the information terminal which was wirelessly connected before the instruction to measure the biological information is received, starts measuring, the wireless connection is established again with the information terminal of the user who started measuring and the measurement data can be transferred. Thus, it is possible to suppress a decrease in convenience caused by connecting wirelessly in advance.

(7) A control method for an information terminal, including a wireless communication unit configured to receive measurement data of biological information from a measurement device configured to measure the biological information and a processor configured to control the wireless communication unit, wherein in a case where an advertise signal wirelessly transmitted from the measurement device has been received, the processor makes a connection request to the measurement device depending on whether or not information indicating a user different from a user of the information terminal is included in the advertise signal.

According to (7), since it is possible to recognize that the user different from the user of the information is currently being measured on the basis of the information terminal indicating the user included in the received advertise signal, it is possible to perform control so as not to perform a connection request by detecting the advertise signal. This can reduce unnecessary processing and reduce power consumption.

(8) A control program for an information terminal, including a wireless communication unit configured to receive measurement data of biological information from a measurement device configured to measure the biological information and a processor configured to control the wireless communication unit, the control program causing the processor to execute processing wherein
in a case where an advertise signal wirelessly transmitted from the measurement device has been received, a connection request is made to the measurement device depending on whether or not information indicating a user different from a user of the information terminal is included in the advertise signal.

According to (8), since it is possible to recognize that the user different from the user of the information terminal is currently being measured on the basis of the information indicating the user included in the received advertise signal, it is possible to perform control so as not to perform a connection request by detecting the advertise signal. This can reduce unnecessary processing and reduce power consumption.

### Advantageous Effects of Invention

According to the present invention, it is possible to provide a measurement device, an information terminal, a control method, and a control program that can reduce power consumption.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a diagram illustrating an information management system 100 including a measurement device 1 of the present invention and information terminals 5a and 5b that performs wireless communication with the measurement device 1.

FIG. 2 is a diagram illustrating a blood pressure monitor 1A that is an example of the measurement device 1.

FIG. 3 is a diagram illustrating an example in which the information terminals 5a and 5b are connected to a network.

FIG. 4 is a block diagram illustrating a configuration of the measurement device 1.

FIG. 5 is a block diagram illustrating a configuration of the information terminal 5.

FIG. 6 is a sequence diagram illustrating an example of the operations of the measurement device 1 and the information terminals 5a, 5b in the information management system 100.

FIG. 7 is a sequence diagram illustrating a modified example of the operations of the measurement device 1 and the information terminals 5a, 5b in the information management system 100.

FIG. 8 is a flow chart illustrating an example of operations in the measurement device 1.

FIG. 9 is a flowchart illustrating connection switching processing.

FIG. 10 is a flow chart illustrating an example of an operation in the information terminals 5a, 5b.

### DESCRIPTION OF EMBODIMENTS

Embodiments according to one aspect of the present invention will be described below based on the drawings.

### § 1 Application Example

### Information Management System 100 Adopting Present Invention

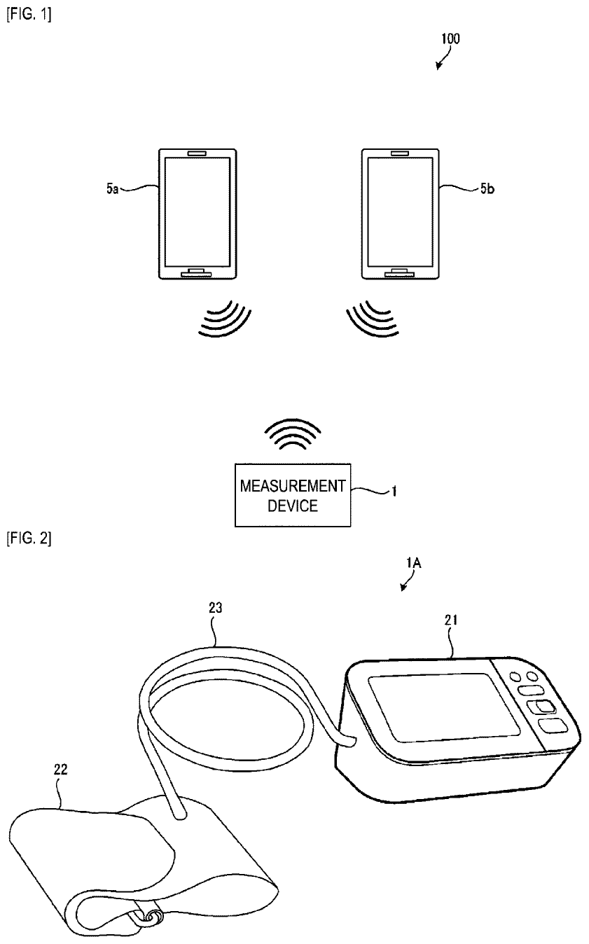

FIG. 1 illustrates an information management system 100 including a measurement device 1 of the present invention and information terminals 5a and 5b that performs wireless communication with the measurement device 1.

The measurement device 1 includes a biological information measurement device that measures biological information such as body weight, body composition, blood pressure, pulse, heart rate, body temperature, blood glucose, or blood oxygen saturation level. The measurement device 1 includes a measurement sensor for measuring a measurement target amount. The measurement target amount of the measurement sensor includes biological information such as body weight, body fat percentage, blood pressure value, pulse rate, heart rate, body temperature, blood glucose value, or blood oxygen saturation level in accordance with the measurement device 1. The measurement device 1 is a non-wearable measurement device. The non-wearable measurement device is a measurement device that is not wearable. The wearable measurement device is a measurement device (e.g., an activity meter) carried by being worn on the body of a user. For example, the measurement device 1 (a non-wearable measurement device) is a measurement device such as a scale, a body composition meter, a body weight-composition meter, or a blood pressure monitor that is used in a state of being installed on a ground or a table. The measurement device 1 transmits measured biological information as measurement data of a user to the information terminals 5a and 5b by wireless communication.

The information terminals 5a and 5b store the measurement data received from the measurement device 1 in a data storage unit in the information terminals 5a and 5b. The information terminals 5a and 5b can perform wireless communication also with an external device other than the measurement device 1 and store information acquired from the external device in the data storage unit in the information terminals 5a and 5b. The information terminals 5a and 5b are each an information processing device that analyzes various types of information acquired from the measurement device 1 and other external devices. The information terminals 5a and 5b are each a terminal having a display, for example, such as a smart phone, a tablet terminal, a laptop computer, a desktop computer, or a wearable terminal. The information terminals 5a and 5b may be set to acquire measurement data from a specific measurement device 1. The specific measurement device 1 from which the measurement data is acquired may be registered in advance in the data storage unit of the information terminals 5a and 5b. Although two information terminals are illustrated in this example, the number of information terminals is not limited thereto, and two or more information terminals may be included.

FIG. 2 is a diagram illustrating a blood pressure monitor 1A that is an example of the measurement device 1. The blood pressure monitor 1A is an example of a biological information measurement device, measures a blood pressure (pressure pulse wave data) of the user, and outputs a measurement result thereof to the user. The blood pressure monitor 1A transmits the measurement result as measurement data of the user to the information terminals 5a and 5b by wireless communication. For example, the blood pressure monitor 1A includes a main body portion 21, a cuff 22 that can be wound around an upper arm of the user, and an air tube 23 that connects the main body portion 21 and the cuff 22. In the example of FIG. 2, the cuff 22 and the main body portion 21 are separated, but the cuff 22 may be integrated with the main body portion 21.

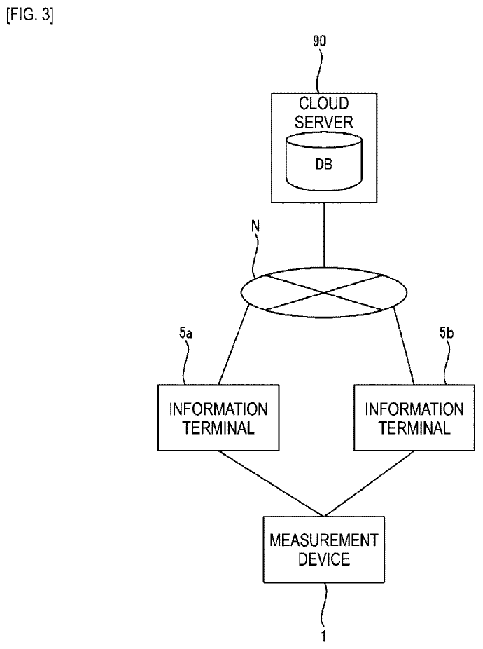

FIG. 3 is a diagram illustrating an example in which the information terminals 5a and 5b are connected to a network. As illustrated in FIG. 3, the information terminals 5a and 5b may be connected to a cloud server 90 via a wide area network N such as the Internet. The information terminals 5a and 5b may transmit the measurement data stored in the information terminal 5 to the cloud server 90 via the wide area network N, and the cloud server 90 may manage the measurement data of the user as a database. The information terminals 5a and 5b may acquire the measurement data managed in the cloud server 90 via the wide area network N and use the acquired measurement data.

### § 2 Configuration Example

### Configuration of Measurement Device 1

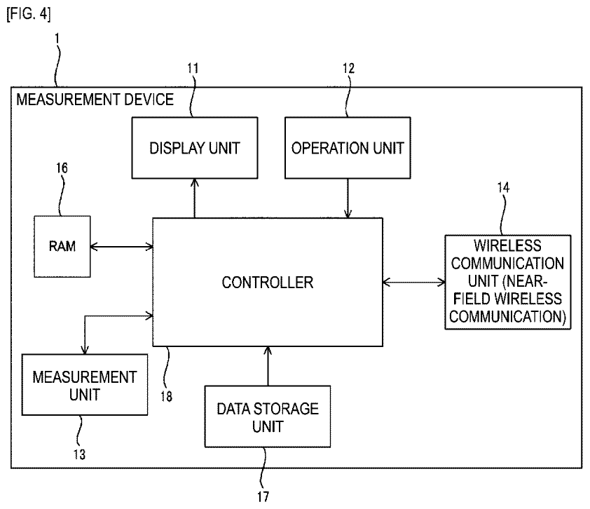

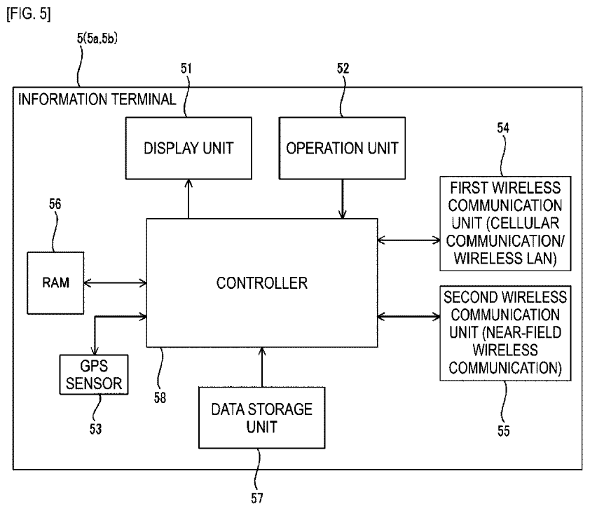

FIG. 4 is a block diagram illustrating a configuration of the measurement device 1. The measurement device 1 includes a display unit 11 that displays various information, an operation unit 12 operated by a user, a measurement unit 13 that measures biological information and the like, and a wireless communication unit 14 that communicates with an external device. The measurement device 1 further includes a RAM 16 that temporarily stores information, a data storage unit 17 that stores information and programs, and a controller 18 that controls the overall operation. The controller 18 is an example of “a control unit” in the present invention.

The display unit 11 includes, for example, a liquid crystal display or an organic electro luminescence (EL) display. The operation unit 12 is a user interface that receives a user operation such as a button or a touch panel. The button includes a button physically disposed at the measurement device 1 or a virtual button displayed on the display unit 11.

The measurement unit 13 measures biological information such as body weight, body composition, blood pressure, pulse, heart rate, body temperature, blood glucose, and blood oxygen saturation level. What to be measured varies in accordance with a measurement target of the measurement device 1.

The wireless communication unit 14 is a communication unit that performs near-range wireless communication, for example, a circuit (module) for performing communication in compliance with Bluetooth Low Energy (registered trademark) (BLE) standard. The wireless communication unit 14 transmits, in a predetermined transmission cycle, an advertise signal for performing wireless communication to an unspecified number of external devices by broadcast communication. The wireless communication unit 14 sends an advertise signal including, for example, the name and attribute information of the measurement device 1. The wireless communication (BLE communication) performed by the wireless communication unit 14 is, for example, communication using 2.4 GHz frequencies. The wireless communication unit 14 transmits measurement data such as biological information measured by the measurement unit 13 to the information terminals 5 through BLE communication.

The RAM 16 includes, for example, a semiconductor device such as a DRAM or an SRAM and temporarily stores information and functions as a work area for the controller 18.

The data storage unit 17 is a recording medium that stores parameters, a control program, measurement data, and the like necessary for implementing a predetermined function. The data storage unit 17 includes, for example, a hard disk drive (HDD) or a semiconductor storage device (SSD).

The controller 18 is a processor that implements a predetermined function by executing the control program. In the present embodiment, for example, health management application software for the measurement device is installed in advance as the control program in the data storage unit 17, and the controller 18 implements the predetermined function by executing the health management application software.

For example, the controller 18 controls the wireless communication unit 14 to transmit an advertise signal for wireless communication (BLE communication) at a predetermined transmission cycle. Specifically, when the measurement device 1 is activated, the controller 18 performs control to transmit an advertise signal. For example, when the power button of the main body portion 21 is turned on in the blood pressure monitor 1A illustrated in FIG. 2, the controller 18 controls the wireless communication unit 14 to start transmission of the advertise signal.

Before receiving an instruction to measure biological information from the user, the controller 18 causes the wireless communication unit 14 to wirelessly transmit an advertise signal and performs control to wirelessly connect to the first information terminal (for example, the information terminal 5a) that has made a connection request in response to the advertise signal. The controller 18 controls the wireless communication unit 14 to stop the transmission of the advertise signal during a period in which the wireless communication between the measurement device 1 and the first information terminal is connected.

During the measuring in which the controller 18 receives an instruction to start measuring the biological information from the user and performs measuring, when the subject who has given the instruction to start measuring the biological information is different from a user a of the first information terminal (for example, the information terminal 5a) to which a controller 58 is currently wirelessly connected, the controller 18 performs control to disconnect the wireless connection with the first information terminal and to wirelessly connect to the second information terminal (for example, the information terminal 5b) of the subject (for example, a user b) who has given the instruction to start measuring the biological information. After the wireless connection with the first information terminal is disconnected and the wireless connection with the second information terminal is established, the controller 18 performs control to wirelessly transmit an advertise signal including information indicating the subject (user b) (for example, “measurement user” information). The controller 58 may further include information indicating that measurement is currently being performed (for example, “measurement flag” information) in the advertise signal and transmit the advertise signal.

The controller 18 performs control to set the signal transmission interval with the first information terminal to a first interval after wirelessly connecting to the first information terminal before receiving an instruction to measure biological information from the user, and to set the signal transmission interval with the information terminal of the subject to a second interval shorter than the first interval when wirelessly transmitting measurement data of biological information to the information terminal of the subject. The “signal transmission interval” is an interval at which a data packet is transmitted, that is, a connection interval. The “second interval” is an interval for normal communication, and is, for example, an interval of 7.5 ms. The “first interval” is longer than the second interval, is the longest interval that can be set, and is an interval of 10 sec, for example.

### Configuration of Information Terminal 5 (5a, 5b)

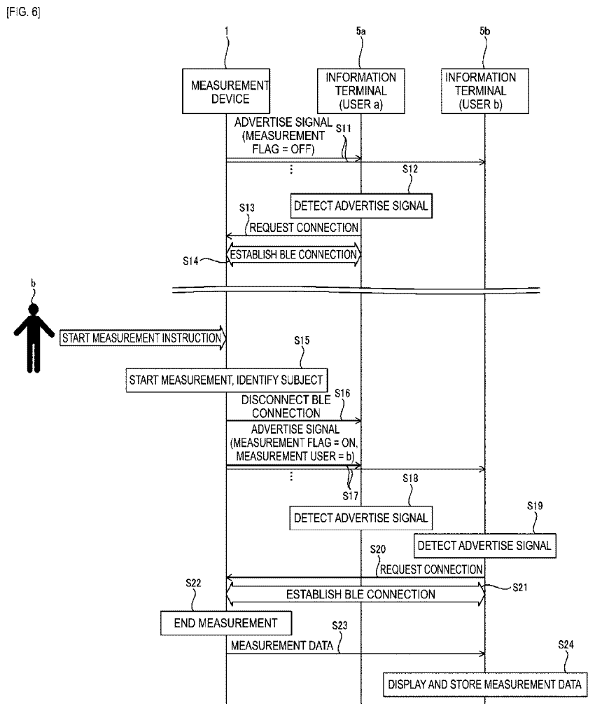

FIG. 5 is a block diagram illustrating a configuration of the information terminals 5 (5a, 5b). The information terminal 5 includes a display unit 51 that displays various types of information, an operation unit 52 that is operated by the user, a global positioning system (GPS) sensor 53 for detecting a position, and a first wireless communication unit 54 and a second wireless communication unit 55 that perform communication with an external device. The information terminal 5 includes a RAM 56 that temporarily stores information, a data storage unit 57 that stores information and programs, and the controller 58 that controls the entire operations.

The display unit 51 includes, for example, a liquid crystal display or an organic electro luminescence (EL) display. The operation unit 52 is a user interface that receives a user operation such as a button or a touch panel. The button includes a button physically disposed at the information terminal 5 or a virtual button displayed on the display unit 51. The GPS sensor 53 is a sensor for detecting the current position of the information terminal 5.

The first wireless communication unit 54 is a communication unit that performs cellular communication, for example, a circuit (module) that can perform communication in compliance with a standard such as 4G, 5G, or long term evolution (LTE: registered trademark). The first wireless communication unit 54 is a communication unit that performs wireless LAN communication, for example, a circuit (module) that can perform communication in compliance with a standard such as Wi-Fi (registered trademark). The second wireless communication unit 55 is a communication unit that performs near-range wireless communication, for example, a circuit (module) that can communicate in compliance with the BLE standard.

The second wireless communication unit 55 acquires the measurement data of the user measured by the measurement device 1, for example, by performing BLE communication with the wireless communication unit 14 of the measurement device 1. The second wireless communication unit 55 receives the advertise signal transmitted from the wireless communication unit 14 of the measurement device 1 by scanning. The second wireless communication unit 55 recognizes the measurement device 1 from the received advertise signal, and transmits a connection request to the measurement device 1 when communication connection is desired. Note that the measurement device 1 waits for a connection request for a predetermined time after transmitting the advertise signal, stops sending the advertise signal upon receiving the connection request within the predetermined time, and switches to one-to-one connection communication with a person requesting the connection.

The RAM 56 includes, for example, a semiconductor device such as a DRAM or an SRAM and temporarily stores information and functions as a work area for the controller 58.

The data storage unit 57 is a recording medium that stores a parameter necessary for implementing a predetermined function, a control program, measurement data acquired from the measurement device 1, and the like. The data storage unit 57 includes, for example, a hard disk drive (HDD) or a semiconductor storage device (SSD).

The controller 58 is a processor that implements a predetermined function by executing the control program. Note that in the present embodiment, for example, health management application software for an information terminal is installed in advance as a control program in the data storage unit 57, and the controller 58 implements the predetermined function by executing this health management application software.

For example, when the health management application software for the information terminal is started, the controller 58 controls the second wireless communication unit 55 to receive the advertise signal by scanning. When the advertise signal from the measurement device 1 is received, the controller 58 controls the second wireless communication unit 55 to transmit a connection request to the measurement device 1. When the connection request is received by the measurement device 1, the communication of the measurement device 1 is switched to one to-one connection communication with the information terminal 5, and the information terminal 5 and the measurement device 1 are put in a state of being BLE-connected.

When the controller 58 receives an advertise signal wirelessly transmitted from the measurement device 1, the controller 58 determines whether or not to transmit a connection request to the measurement device 1 depending on whether or not information indicating a user different from the user of the information terminal 5 (for example, “measurement user” information) is included in the advertise signal.

### § 3 Operation Example

### Operations of Information Management System 100

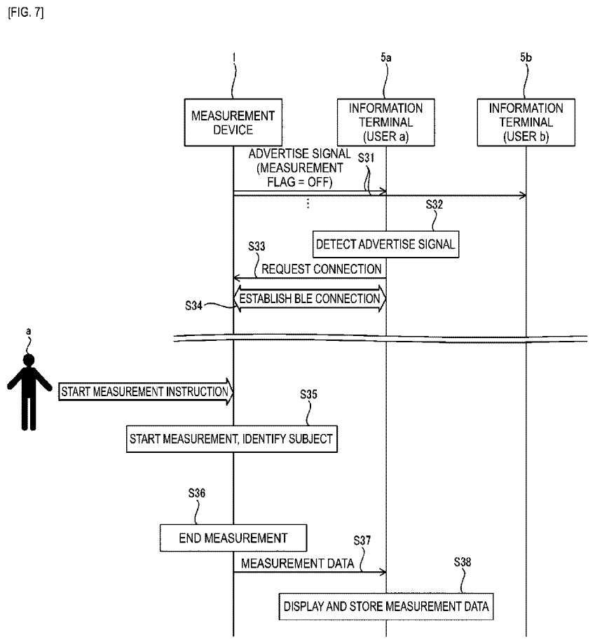

Next, with reference to FIG. 6, operations of the information management system 100 will be described. FIG. 6 is a sequence diagram illustrating an example of the operations of the measurement device 1 and the information terminal 5 (5a, 5b) in the information management system 100. It is assumed that the information terminal 5a is an information terminal owned by the user a, and the information terminal 5b is an information terminal owned by the user b. It is also assumed that the information terminals 5a and 5b have been paired with the measurement device 1.

First, when the power of the measurement device 1 is turned on, for example, the measurement device 1 executes a health management application and starts transmitting an advertise signal for wireless communication (BLE communication) (step S11). The measurement device 1 transmits the advertise signal at a predetermined cycle and transmits the advertise signal including, for example, “measurement flag=OFF” indicating that the measurement in the measurement device 1 has not been started yet.

Next, for example, it is assumed that the information terminal 5a has performed scanning by executing the health management application and has detected an advertise signal from the measurement device 1 (step S12). When the information terminal 5a detects the advertise signal from the measurement device 1, the information terminal 5a transmits a connection request signal for requesting a communication connection to the measurement device 1 (step S13).

Next, when the measurement device 1 receives the connection request from the information terminal 5a, the measurement device 1 stops transmitting the advertise signal and starts one to-one connection communication with the information terminal 5a. Accordingly, the measurement device 1 and the information terminal 5a are BLE-connected (step S14).

Next, for example, it is assumed that the user b who owns the information terminal 5b starts measurement of biological information of the user b using the measurement device 1. The user b presses, for example, a start measurement button of the measurement device 1 to issue an instruction to start measurement.

Next, the measurement device 1 starts measuring measurement data in response to the instruction to start measuring and identifies the subject (step S15). The identification of the subject may be performed by identification based on a user selection via a button operation by the subject or identification based on a measurement value (obtained at an initial stage). The term “based on a measurement value” includes, for example, a case where the measurement value is specified by comparison with a value stored as a previous measurement value of the subject.

Next, the measurement device 1 determines whether or not the identified subject matches the user a of the information terminal 5a currently performing BLE communication, and if the subject is the user b and is different from the user a of the information terminal 5a performing BLE communication as in this example, the measurement device 1 disconnects the BLE connection performed with the information terminal 5a (step S16).

Next, the measurement device 1 starts transmission of an advertise signal for wireless communication (BLE communication) (step S17). The measurement device 1 transmits the advertise signal at a predetermined cycle and transmits, for example, “measurement flag=ON” indicating that the measurement device 1 is currently measuring data and “measurement user=b” indicating that the measurer who is measuring biological information is the user b, which are included in the advertise signal.

Next, it is assumed that the information terminal 5a performs scanning by executing the health management application and detects an advertise signal from the measurement device 1 (step S18). In a similar manner, the information terminal 5b also detects an advertise signal from the measurement device 1 (step S19).

The information terminal 5b detects “measurement user=b” included in the advertise signal from the measurement device 1, determines that the user b who owns the information terminal 5b is measuring biological information, and transmits a connection request signal for requesting a communication connection to the measurement device 1 (step S20). On the other hand, the information terminal 5a detects “measurement user=b” included in the advertise signal, determines that a user different from the owner of the information terminal 5a is measuring biological information, and does not make a connection request to the measurement device 1.

Next, the measurement device 1 receives the connection request from the information terminal 5b, stops transmitting the advertise signal, and starts one-to-one connection communication with the information terminal 5b. Accordingly, the measurement device 1 and the information terminal 5b are BLE-connected (step S21).

Next, when the measurement device 1 ends the measurement of the measurement data (step S22), the measurement device 1 transmits the measured measurement data to the information terminal 5b by BLE communication (step S23).

Next, the information terminal 5b displays the measurement data received from the measurement device 1 on the display unit 51 and stores the measurement data in the date storage unit 57 (step S24). Thereafter, the BLE connection between the information terminal 5b and the measurement device 1 is maintained until, for example, the next start measurement instruction is issued and it is determined that the subject and the user b of the information terminal 5b currently performing BLE communication are different from each other.

### Modified Example of Operations of Information Management System 100

Next, with reference to FIG. 7, a modified example of operations of the information management system 100 will be described. FIG. 7 is a sequence diagram illustrating a modified example of the operations of the measurement device 1 and the information terminal 5 (5a, 5b) in the information management system 100. As in the operation example of FIG. 6, it is assumed that the information terminal 5a of the user a and the information terminal 5b of the user b have been paired with the measurement device 1.

The processing from step S31 to step S34 in the present modified example illustrated in FIG. 7 is similar to the processing from step S11 to step S14 of the operation example illustrated in FIG. 6. Thus, the description of the processing of these steps is omitted.

Next, for example, it is assumed that the user a who owns the information terminal 5a starts measurement of biological information of the user a using the measurement device 1. The user a presses, for example, a start measurement button of the measurement device 1 to issue an instruction to start measurement.

Next, the measurement device 1 starts measuring measurement data in response to the instruction to start measuring and identifies the subject (step S35). As in the case of step S15 in the operation example of FIG. 6 described above, the identification of the subject may be performed by identification based on a user selection via a button operation by the subject or identification based on a measurement value (obtained at an initial stage). The measurement device 1 determines whether or not the identified subject matches the user a of the information terminal 5a currently performing BLE communication, and if the subject is the user a and matches the user a of the information terminal 5a performing BLE communication as in this example, the measurement device 1 maintains the state of the BLE connection performed with the information terminal 5a.

Next, when the measurement device 1 ends the measurement of the measurement data (step S36), the measurement device 1 transmits the measured measurement data to the information terminal 5a by BLE communication (step S37).

Next, the information terminal 5a displays the measurement data received from the measurement device 1 on the display unit 51 and stores the measurement data in the date storage unit 57 (step S38). Thereafter, the BLE connection between the information terminal 5a and the measurement device 1 is maintained until, for example, the next start measurement instruction is issued and it is determined that the subject and the user a of the information terminal 5a currently performing BLE communication are different from each other.

### Operations of Measurement Device 1

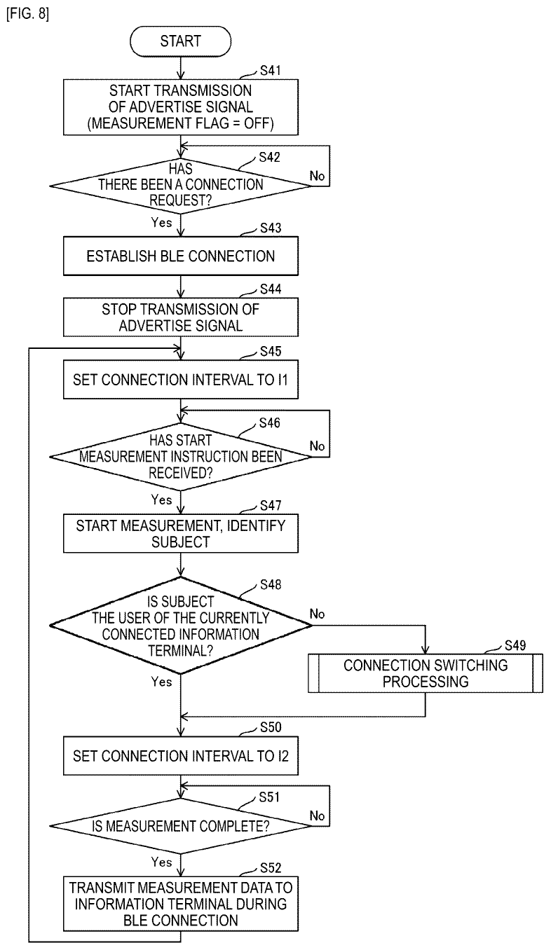

Next, operations of the measurement device 1 will be described with reference to FIG. 8. FIG. 8 is a flow chart illustrating an example of operations in the measurement device 1. The processing of the present operations is started when the power of the measurement device 1 is turned on, for example.

First, when the power is turned on, for example, the measurement device 1 executes a health management application and starts transmitting an advertise signal for BLE communication (step S41). The measurement device 1 transmits the advertise signal including, for example, “measurement flag=OFF” indicating that the measurement in the measurement device 1 has not been started yet. The measurement device 1 repeatedly transmits the advertise signal at a predetermined cycle.

Next, the measurement device 1 determines whether or not there is a connection request from, for example, the paired information terminal 5a or 5b or the like in response to the transmitted advertise signal (step S42). In a case where there is no connection request (step S42: No), the measurement device 1 repeats the processing of step S42.

In step S42, when there is a connection request (step S42: Yes), the measurement device 1 starts communication with the information terminal 5a or 5b or the like that made the connection request by BLE connection (step S43).

Next, the measurement device 1 stops the transmission of the advertise signal started in step S41 (step S44).

Next, the measurement device 1 sets the Connection Interval with the information terminal 5a or 5b or the like that started the BLE communication in step S43 to “I1” (step S45). I1 is a transmission interval of packets in BLE communication with the information terminal 5a or 5b or the like, which is performed when the measurement device 1 has not yet started measuring data (when the measurement flag=OFF). For example, I1 is the longest interval that can be set in the measurement device 1. I1 is an example of the “first interval” of the present invention.

Next, the measurement device 1 determines whether or not an instruction to start measuring biological information by the measurement device 1 has been received from the user (step S46). In a case where an instruction to start measuring has not been received from the user (step S46: No), the measurement device 1 repeats the processing of step S46.

In step S46, in a case where an instruction to start the measuring has been received from the user (step S46: Yes), the measurement device 1 starts measuring the measurement data and identifies the user who instructed the start of the measuring, that is, the subject in the present measuring (step S47). As in the case of step S15 of FIG. 6 described above, the identification of the subject may be performed by identification based on a user selection via a button operation by the subject or identification based on a measurement value (obtained at an initial stage).

Next, the measurement device 1 determines whether or not the subject identified in step S47 is the user a or b or the like of the information terminal 5a or 5b or the like currently connected by BLE (step S48).

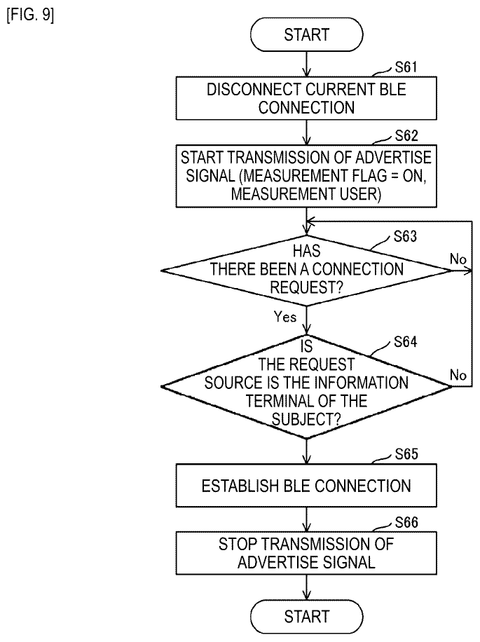

In step S48, in a case where the subject is not the user of the information terminal currently connected by BLE (step S48: No), the measurement device 1 proceeds to “connection switching processing” illustrated in FIG. 9 and switches the information terminal to be connected by BLE (step S49). Then, the measurement device 1 switches the connection and sets the Connection Interval with the information terminal 5a or 5b or the like that started the BLE communication to “I2” (step S50). I2 is a transmission interval of packets in BLE communication with the information terminal 5a or 5b or the like, which is performed when the measurement device 1 is currently measuring data (when the measurement flag=ON). For example, I2 is a transmission interval used in normal BLE communication. I2 is an example of the “second interval” of the present invention.

On the other hand, in step S48, in a case where the subject is the user of the information terminal currently BLE-connected (step S48: Yes), the measurement device 1 sets the Connection Interval with the BLE-connected information terminal 5a or 5b or the like to “I2” while maintaining the current BLE connection (step S50).

Next, the measurement device 1 determines whether or not the data measurement started in step S46 has ended (step S51). In a case where measurement has not ended (step S51: No), the measurement device 1 repeats the processing of step S51.

In step S51, in a case where measurement has ended (step S51: Yes), the measurement device 1 transmits the measurement data to the information terminals BLE-connected (step S52). After transmitting the measurement data to the information terminals, the measurement device 1 returns to step S45, sets the Connection Interval with the BLE-connected information terminal 5a or 5b or the like to “I1” and repeats each item of processing.

In the processing example described above, the measurement device 1 executes the processing of setting the Connection Interval to “I2” before determining whether or not the measurement has ended (step 51), but no such limitation is intended. For example, the measurement device 1 may execute the processing of setting the Connection Interval to “I2” after determining whether or not the measurement has ended.

### Connection Switching Processing

FIG. 9 is a flowchart illustrating the “connection switching processing” in step S49 of FIG. 8. In a case where the subject and the user of the information terminal currently BLE-connected are different, first, the measurement device 1 disconnects the current BLE connection with the information terminal 5a or 5b or the like (step S61).

Next, the measurement device 1 starts transmission of an advertise signal for BLE communication (step S62). In this case, the measurement device 1 transmits, for example, information of “measurement flag=ON” indicating that the measurement device 1 is currently measuring data and information of “measurement user” indicating the measurer who is measuring biological information, which are included in the advertise signal.

Next, the measurement device 1 determines whether or not there is a connection request from the information terminal 5a or 5b or the like in response to the advertise signal transmitted in step 62 (step S63). In a case where there is no connection request (step S63: No), the measurement device 1 repeats the processing of step S63.

In step S63, when there is a connection request (step S63: Yes), the measurement device 1 determines whether or not the request source of the connection request is the information terminal 5a or 5b or the like of the subject (step S64).

In step S64, in a case where the request source of the connection request is not the information terminal 5a or 5b or the like of the subject (step S64: No), the measurement device 1 returns to step S63 and repeats each item of processing. In step S64, in a case where the request source of the connection request is the information terminal 5a or 5b or the like of the subject (step S64: Yes), the measurement device 1 starts communication with the information terminal 5a or 5b or the like that made the connection request by BLE connection (step S65).

Next, the measurement device 1 stops the transmission of the advertise signal started in step S62 (step S66) and ends the connection switching processing.

As described above, the measurement device 1 wirelessly transmits an advertise signal before an instruction to measure biological information and performs control to wirelessly connect to, for example, the information terminal 5a that made a connection request in response to the advertise signal. In addition, in a case where the subject whose biological information is to be measured is different from the user of the information terminal 5a to which the measurement device 1 is wirelessly connected at the time that biological information is measured, the measurement device 1 performs control to disconnect the wireless connection to the information terminal 5a to which the measurement device 1 is wirelessly connected and to wirelessly connect to the information terminal 5b of the subject. According to this configuration, by transmitting the advertise signal and wirelessly connecting to the information terminal 5a that made the connection request before receiving the instruction to measure the biological information, it is possible to reduce power consumption by stopping the transmission of an advertise signal with a transmission interval that cannot be made too long due to the specifications. In this case, although it is necessary to transmit and receive packets at intervals of the Connection Interval in order to maintain the wireless connection, the Connection Interval can be made longer than the transmission interval of the advertise signal, and thus the total power consumption can be reduced. In addition, in a case where the user a of the information terminal 5a, which was wirelessly connected before the instruction to measure the biological information is received, starts measuring the biological information, the measurement data can be transferred to the information terminal 5a of the user a through the wireless connection, and thus, the convenience can be improved. In a case where the user b, who is different from the user a of the information terminal 5a which was wirelessly connected before the instruction to measure the biological information is received, starts measuring, the wireless connection is established again with the information terminal 5b of the user b who started measuring and the measurement data can be transferred. Thus, it is possible to suppress a decrease in convenience caused by connecting wirelessly in advance.

When the measurement device 1 is wirelessly connected to the information terminal 5b of the user b who has started measuring the biological information, the measurement device 1 wirelessly transmits an advertise signal including information indicating the user b who is the subject (measurement user=b). According to this configuration, the information terminal 5a of the user a, who is different from the subject, can recognize from the advertise signal that the user b different from the user a of the information terminal 5a is being measured, and thus can perform control so as not to make a connection request even if an advertise signal is detected. Thus, it is possible to suppress competition between the information terminal 5b of the subject (user b) and the information terminal 5a of the user a different from the subject.

For example, when the measurement device 1 is wirelessly connected to, for example, the information terminal 5a before measuring the biological information, the measurement device 1 sets the Connection Interval with the information terminal 5a to the first interval, and when the measurement device 1 wirelessly transmits the measurement data of the biological information to the information terminal 5a or 5b of the subject, the measurement device 1 sets the Connection Interval with the information terminal 5a or 5b of the subject to the second interval which is shorter than the first interval. According to this configuration, the first interval for transmitting the signal to the wirelessly connected information terminal 5a before measuring the biological information is set to be longer than the second interval for transmitting the signal when wirelessly transmitting the measurement data of the biological information to the information terminal 5a or 5b of the subject, so that the number of times of signal transmission can be reduced, and power consumption can be reduced.

### Operation of Information Terminal 5 (5a, 5b)

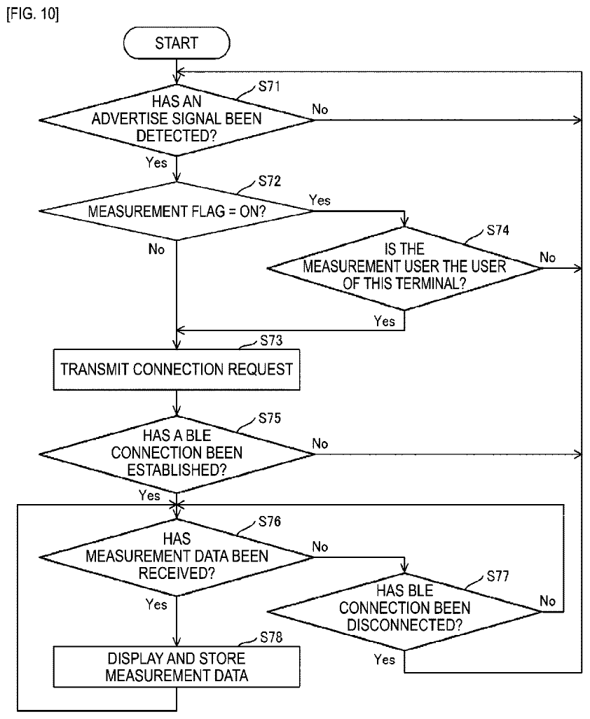

Next, the operation of the information terminal 5 (5a, 5b) will be described with reference to FIG. 10. FIG. 10 is a flow chart illustrating an example of an operation in the information terminal 5 (5a, 5b). The processing of the present operation is started when the health management application of the information terminal 5 (5a, 5b) is run.

First, the information terminal 5 determines whether or not an advertise signal from the measurement device 1 has been detected (step S71). In a case where an advertise signal has not been detected (step S71: No), the information terminal 5 repeats the processing of step S71.

In step S71, in a case where the advertise signal has been detected (step S71: Yes), the information terminal 5 determines whether or not the information of the “measurement flag” included in the advertise signal is “measurement flag=ON” (step S72). The information of “measurement flag=ON” is information indicating that the measurement device 1 is currently measuring data as described above.

In step S72, in a case where “measurement flag=ON” is not the case, that is, if the measurement device 1 is not currently measuring data (step S72: No), the information terminal 5 transmits a connection request to the measurement device 1 (step S73).

In step S72, in a case where “measurement flag=ON” is the case, that is, if the measurement device 1 is currently measuring (step S72: Yes), the information terminal 5 determines whether or not the information of the “measurement user” included in the advertise signal is information indicating the user of the information terminal 5 (step S74).

In step S74, in a case where the information of the “measurement user” is information indicating the user of the information terminal 5 (step S74: Yes), the information terminal 5 transmits a connection request to the measurement device 1 (step S73). In step S74, in a case where the information of the “measurement user” is not information indicating the user of the information terminal 5 (step S74: No), the information terminal 5 returns to step S71 and repeats each item of processing.

Next, the information terminal 5 determines whether or not a BLE connection has been established with the measurement device 1 (step S75). In a case where a BLE connection with the measurement device 1 has not been established (step S75: No), the information terminal 5 returns to step S71 and repeats each item of processing.

In step S75, in a case where a BLE connection with the measurement device 1 has been established (step S75: Yes), the information terminal 5 determines whether or not the measurement data has been received from the measurement device 1 (step S76). The measurement data is transmitted when the measurement in the measurement device 1 is completed.

In step S76, in a case where the measurement data has not been received from the measurement device 1 (step S76: No), the information terminal 5 determines whether or not the BLE connection with the measurement device 1 has been disconnected (step S77). In a case where the BLE connection with the measurement device 1 has not been disconnected (step S77: No), the information terminal 5 returns to step S76 and repeats each item of processing. In a case where the BLE connection with the measurement device 1 has been disconnected (step S77: Yes), the information terminal 5 returns to step S71 and repeats each item of processing.

In step S76, in a case where the measurement data has been received from the measurement device 1 (step S76: Yes), the information terminal 5 displays the received measurement data on the display unit 51 and stores the measurement data in the date storage unit 57 (step S78). After displaying and storing the measurement data, the information terminal 5 returns to step S76 and repeats each item of processing.

As described above, in a case where the information terminal 5 has received the advertise signal wirelessly transmitted from the measurement device 1, the information terminal 5 makes a connection request to the measurement device 1 depending on whether or not the advertise signal includes information indicating the user b (measurement user=b) different from the user a of the information terminal 5a, for example. According to this configuration, since it is possible to recognize that the user b different from the user a of the information terminal 5a is currently being measured on the basis of the information indicating the user b (measurement user=b) included in the received advertise signal, it is possible to perform control so as not to perform a connection request by detecting the advertise signal. This can reduce unnecessary control processing and reduce power consumption.

### § 4. Modifications

While the embodiment of the present invention is described in detail above, the above description is merely illustrative of the present invention in all respects. Various modifications and variations can be made without departing from the scope of the present invention. For example, the following changes can be made. Note that in the following, the same reference signs are used for components that are the same as those of the above-described embodiment, and descriptions thereof are omitted as appropriate. The following modified examples can be combined as appropriate.

In the embodiments described above, a case has been described in which the information of the “measurement flag” indicating that the measurement device 1 is currently measuring data is included in the advertise signal and transmitted, but no such limitation is intended. The measurement device 1 may not include the information of the “measurement flag” in the advertise signal. In this case, the information terminal that receives the advertise signal may determine whether or not the measurement device 1 is currently measuring data, for example, on the basis of the presence or absence of the “measurement user” included in the advertise signal.

### § 5 Program

Note that the control method for the measurement device 1 or the information terminal 5 explained in the above-described embodiment can be realized by a computer executing a control program prepared in advance. The control program is recorded in a computer-readable storage medium and is executed by being read from the storage medium. The control program may be provided in a form of being stored in a non-transitory storage medium such as a flash memory or may be provided via a network such as the Internet. The computer that executes the control program may be included in the measurement device 1 or the information terminal 5, may be included in an electronic device such as a smart phone, a tablet terminal, or a personal computer that can communicate with the measurement device 1 or the information terminal 5, or may be included in a server device that can communicate with the measurement device 1, the information terminal 5, and the electronic device.

Although various embodiments are described above, it will be obvious that the present invention is not limited to such examples. It will be apparent to those skilled in the art that various changes and modifications can be made within the scope of the claims, and it is understood that these naturally belong to the technical scope of the present invention. In addition, components of the above-described embodiments may be combined as appropriate without departing from the spirit of the invention.

Note that the present application is based on Japanese Patent Application (Japanese Patent Application No. 2023-093847) filed on Jun. 7, 2023, the contents of which are incorporated herein by reference.

### REFERENCE NUMERALS LIST

1 Measurement device
1A Blood pressure monitor
5, 5a, 5b Information terminal
11, 51 Display unit
12, 52 Operation unit
13 Measurement unit
14 Wireless communication unit
16, 56 RAM
17, 57 Data storage unit
18, 58 Controller
21 Main body portion
22 Cuff
23 Air tube
53 GPS sensor
54 First wireless communication unit
55 Second wireless communication unit
90 Cloud server
100 Information management system

1-9. (canceled)

10. A measurement device that can measure biological information, comprising:
a wireless communication unit configured to transmit measurement data of the biological information to an information terminal; and
a control unit configured to control the wireless communication unit, wherein:
before an instruction to measure the biological information, the control unit performs control to wirelessly transmit an advertise signal and wirelessly connect to a first information terminal that made a connection request in response to the advertise signal, and
at a time of measuring the biological information, in a case where a subject is different from a user of the first information terminal, the control unit performs control to disconnect from the first information terminal and wirelessly connect to a second information terminal of the subject.

11. The measurement device according to claim 10, wherein:
at a time of wirelessly connecting to the second information terminal, the control unit wirelessly transmits an advertise signal including information indicating the subject.

12. The measurement device according to claim 10, wherein:
when a wireless connection with the first information terminal is established before measurement of the biological information, the control unit sets a signal transmission interval with the first information terminal to a first interval, and
at a time of wirelessly transmitting measurement data of the biological information to an information terminal of the subject, the control unit sets a signal transmission interval with an information terminal of the subject to a second interval shorter than the first interval.

13. The measurement device according to claim 11, wherein:
when a wireless connection with the first information terminal is established before measurement of the biological information, the control unit sets a signal transmission interval with the first information terminal to a first interval, and
at a time of wirelessly transmitting measurement data of the biological information to an information terminal of the subject, the control unit sets a signal transmission interval with an information terminal of the subject to a second interval shorter than the first interval.

14. An information terminal that can wirelessly communicate with a measurement device that can measure biological information, comprising:
a wireless communication unit configured to receive measurement data of the biological information from the measurement device; and
a control unit configured to control the wireless communication unit, wherein:
in a case where an advertise signal wirelessly transmitted from the measurement device has been received, the control unit makes a connection request to the measurement device depending on whether or not information indicating a user different from a user of the information terminal is included in the advertise signal as information indicating a subject.

15. A control method for a measurement device that can measure biological information and includes a wireless communication unit configured to transmit measurement data of the biological information to an information terminal and a processor configured to control the wireless communication unit, wherein:
before an instruction to measure the biological information, the processor performs control to wirelessly transmit an advertise signal and wirelessly connect to a first information terminal that made a connection request in response to the advertise signal; and
at a time of measuring the biological information, in a case where a subject is different from a user of the first information terminal, the processor performs control to disconnect from the first information terminal and wirelessly connect to a second information terminal of the subject.

16. A control program for a measurement device that can measure biological information and includes a wireless communication unit configured to transmit measurement data of the biological information to an information terminal and a processor configured to control the wireless communication unit, the control program causing the processor to execute processing wherein:
before an instruction to measure the biological information, control is performed to wirelessly transmit an advertise signal and wirelessly connect to a first information terminal that made a connection request in response to the advertise signal; and
at a time of measuring the biological information, in a case where a subject is different from a user of the first information terminal, control is performed to disconnect from the first information terminal and wirelessly connect to a second information terminal of the subject.

17. A control method for an information terminal including a wireless communication unit configured to receive measurement data of biological information from a measurement device configured to measure the biological information and a processor configured to control the wireless communication unit, wherein:
in a case where an advertise signal wirelessly transmitted from the measurement device has been received, the processor makes a connection request to the measurement device depending on whether or not information indicating a user different from a user of the information terminal is included in the advertise signal as information indicating a subject.

18. A control program for an information terminal including a wireless communication unit configured to receive measurement data of biological information from a measurement device configured to measure the biological information and a processor configured to control the wireless communication unit, the control program causing the processor to execute processing wherein:
in a case where an advertise signal wirelessly transmitted from the measurement device has been received, a connection request is made to the measurement device depending on whether or not information indicating a user different from a user of the information terminal is included in the advertise signal as information indicating a subject.
